(19) United States
(12) Patent Application Publication
(10) Pub. No.: US 2026/0021048 A1
(43) Pub. Date: Jan. 22, 2026

(54) CONTROLLED RELEASE PHARMACEUTICAL COMPOSITION OF SELEXIPAG OR IT'S ACTIVE METABOLITE

(71) Applicants: Danielle Marie Le Roux, West Milford, NJ (US); Jayendrakumar Dasharathlal Patel, Robbinsville, NJ (US)

(72) Inventors: Danielle Marie Le Roux, West Milford, NJ (US); Jayendrakumar Dasharathlal Patel, Robbinsville, NJ (US)

(21) Appl. No.: 19/292,221

(22) Filed: Aug. 6, 2025

Related U.S. Application Data
(63) Continuation-in-part of application No. 17/428,273, filed on Jul. 5, 2022, now Pat. No. 12,427,145.

(30) Foreign Application Priority Data
Feb. 3, 2019 (IN) 201921004220

(51) Int. Cl. A61K 9/20; A61K 9/28; A61K 31/4965

(52) U.S. Cl. CPC A61K 9/2077; A61K 9/2009; A61K 9/2018; A61K 9/2054; A61K 9/2086; A61K 9/2853; A61K 9/2866; A61K 9/2893; A61K 31/4965

(57) ABSTRACT

This invention provides a method for treating a pulmonary arterial hypertension with reduced incident of side effect using a controlled release pharmaceutical composition of selexipag or its active metabolite, wherein the controlled release pharmaceutical composition of selexipag or its active metabolite following oral administration with an alcoholic beverage reduces incidence of at least one side effect associated with the selexipag or its active metabolite compared to the immediate-release dosage form comprising the same amount of the selexipag or active metabolite thereof.

### CROSS REFERENCE TO RELATED-APPLICATIONS

This U.S. patent application is a Continuation-In-Part of U.S. patent application Ser. No. 17/428,273, filed on Jul. 5, 2022, which is a National Stage Entry of PCT application number: PCT/IB2020/050820, filed on Feb. 2, 2020, which claim priority from Indian provisional patent application no. 201921004220, filed on Feb. 3, 2019, the disclosures of which are incorporated herein by reference in their entireties.

### FIELD OF THE INVENTION

The present invention is relates to a controlled release pharmaceutical composition of Selexipag or its active metabolite having improved tolerability by reduced incident of side effects, to a process for preparing the pharmaceutical composition, and to a method of treating a Pulmonary Arterial Hypertension (PAH) with reduced incident of side effect, comprising administering the controlled release pharmaceutical composition to a mammal, including a human patient, in need of such treatment. The present invention also relates to a controlled release pharmaceutical composition of Selexipag or its active metabolite that provide bi-phasic release rate of selexipag or its active metabolite. The present invention also relates to a controlled release pharmaceutical composition of Selexipag or its active metabolite that affords protection against alcohol dose dumping following oral administration with an alcoholic beverage.

### BACKGROUND OF THE INVENTION

Pulmonary Arterial Hypertension (PAH) is a hemodynamic and pathophysiological condition affecting the pulmonary arterioles and characterized by progressive increases in pulmonary vascular resistance and pulmonary artery pressure, ultimately leading to right heart failure and premature death. Recent therapeutic options have significantly improved the long-term outcome of patients with PAH, but PAH remains a disease with a poor prognosis.

Reduced expression of prostacyclin synthases in the lung and reduced levels of prostacyclin are key features of PAH. Prostacyclin is produced by endothelial cells from prostaglandin H2 (PGH2) by the enzyme prostacyclin synthase. Prostacyclin is a potent vasodilator and also has anti-proliferative, antithrombotic, and anti-inflammatory effects. As PAH is associated with vasoconstriction, proliferation, and thrombosis, there is a strong rationale for using prostacyclin treatment. Restoration of IP receptor signalling using prostacyclin receptor (IP receptor) agonists is an effective strategy in the treatment of the disease.

Selexipag, previously also known as NS-304 or ACT-293987, is a potent orally available and highly selective long acting non-prostanoid prostaglandin 12 (PGI-2) receptor agonist, and has been approved in the Europe and in the United State and marketed by Actelion for the treatment of pulmonary arterial hypertension under brand name UPTRAVI®. As a prodrug, it is in vivo transformed by the liver to its active metabolite.

Selexipag and processes for its preparation are disclosed in WO 2002/088084 and U.S. Pat. No. 7,205,302. WO 2010/150865 discloses three crystalline forms of Selexipag, designated form I, form II and form III. It is described that these forms were obtained either in pure form or as binary mixtures. The crystalline forms of WO 2010/150865 are described as useful for the preparation of pharmaceutical formulations and oral dosage forms, such as tablets comprising crystalline Selexipag, are described. WO 2017/121806 discloses an amorphous solid dispersion comprising Selexipag and methods for preparing the same. American Journal of Cardiovascular Drugs, 2015; 15(3): 195-203, discloses Pharmacokinetics and Tolerability of the Novel Oral Prostacyclin IP Receptor Agonist Selexipag.

Selexipag is rapidly absorbed after oral administration and hydrolyzed to the pharmacologically more active metabolite ACT-333679. The ACT-333679 is considered as the major contributor to the overall activity of the drug. A chemical structure of the Selexipag and its active metabolite ACT-333679 is as follow:

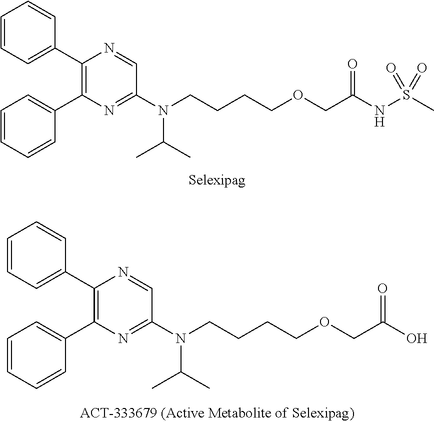 [SMILES: CC(C)N(CCCCOCC(=O)NS(C)(=O)=O)c1cnc(-c2ccccc2)c(-c2ccccc2)n1 ; CC(C)N(CCCCOCC(=O)O)c1cnc(-c2ccccc2)c(-c2ccccc2)n1]

Selexipag is currently sold under brand name UPTRAVI® and is available in a conventional immediate release dosage form. After an oral administration of conventional immediate release dosage form of Selexipag, i.e. UPTRAVI®, it absorbs rapidly and Cmax (Maximum plasma concentration) of Selexipag and its active metabolite are reached within about 1 hour and 2.75 hours, respectively, in fasting condition. However, many of the adverse events has been observed during the oral administration of conventional immediate release dosage form of the Selexipag and they are more worst in a fasting condition relative to a fed condition (Refer “medication guide of UPTRAVI®” as well as “Pharmacokinetics and Tolerability of the Novel Oral Prostacyclin IP Receptor Agonist Selexipag, Am. J. Cardiovasc Drugs. 2015; 15(3): 195-203”).

Adverse events with onset after start of treatment are includes headache, nausea and vomiting. Due to the adverse event, i.e. issue of tolerability, therapy with the UPTRAVI® containing the Selexipag in immediate release form is recommended to start with 200 μg dose followed by increase the dose in increment of 200 μg twice daily, usually at weekly interval, to the highest tolerated dose upto 1600 μg twice daily. Overall, a non-tolerable most frequently reported adverse event is Headache with increasing frequency and intensity at single doses beyond 200 μg, particularly beyond 400 μg. As discussed in the “Pharmacokinetics and Tolerability of the Novel Oral Prostacyclin IP Receptor Agonist Selexipag” (Am. J. Cardiovasc Drugs. 2015; 15(3): 195-203”), 83% subject (5 out of 6 subjects), 33% subject (2 out of 6 subjects) and 17% subject (1 out of 6 subjects) reported Headache after administration of 800 μg, 600 μg and 200 μg Selexipag, respectively, in fasting condition. Hence, adverse events, which if severe, may lead to discontinuation of Therapy with the Selexipag. “Medical Review” section of UPTRAVI® submitted to USFDA disclosed that a total of 24% subjects (52 subjects out of 218 subjects) of the Selexipag treated patients had at least 1 adverse event leading to discontinuation of study drug in study AC-065A303.

As per the medication guide of UPTRAVI®, the most common adverse events reported more frequently after the oral administration of conventional immediate release dosage form of the Selexipag includes Headache in 65% subjects, Diarrhea in 42% subjects, Jaw pain in 26% subjects, Nausea in 33% subjects, Myalgia in 16% subjects, Vomiting in 18% subjects, Pain in Extremity in 17% subjects, Flushing in 12% subjects, Arthralgia in 11% subjects and Rash in 11% subjects out of 575 subjects. Interesting fact is that the adverse event is associated with high peak-trough fluctuation (plasma blood concentration) of Selexipag. This fact is supported by clinical study of UPTRAVI® in terms of differentiation in adverse events in Fed state compared to Fasting state. Compared to Fasting state, pharmacokinetic parameter Cmax is decreased 30% and Tmax extended 2.5 times in Fed state that leads to less adverse events observed when the Selexipag is taken after food (in Fed state). This explicitly indicate that slower rate of Selexipag absorption [Lower Cmax along with Extended Tmax (2.5 times longer)] leads to lower incident of adverse events, indicating that Controlled release dosage form of Selexipag is “Ideal Therapy” compared to conventional immediate release therapy. Therefore, present invention disclosed a controlled release formulation of Selexipag that exhibits significant advantages over the convention immediate release formulation of the Selexipag (UPTRAVI®) of the prior arts. The innovative controlled release formulation of the Selexipag improve a tolerability by reducing an incidence of side effects compared to the conventional immediate release formulation of the Selexipag (UPTRAVI®) as disclosed in the prior arts.

### SUMMARY OF THE INVENTION

Preferred embodiment of this invention provides a controlled release pharmaceutical composition of a Selexipag or its active metabolite that improve tolerability by reducing incidence of side effect associated with the Selexipag or its active metabolite contained in the composition, wherein the composition after administration to a mammal in fasting condition providing a mean or median Tmax (time to reach maximum plasma concentration)
a. of at least greater than 2 hours for the Selexipag, and/or
b. of at least greater than 3.5 hours for the active metabolite of the Selexipag.

Preferred embodiment of this invention provide a controlled release pharmaceutical composition of a Selexipag or its active metabolite that improve tolerability by reducing incidence of side effect associated with the Selexipag or its active metabolite contained in the composition, wherein the composition comprising a release rate controlling ingredient in addition to the Selexipag or its active metabolite that control the releases of the Selexipag or its active metabolite at a rate such that a mean or median Tmax (time to reach maximum plasma concentration) attained after administration of the pharmaceutical composition to mammal in fasting condition is
a. at least greater than about 2.5 hours for the Selexipag, and/or
b. at least greater than about 4 hours for the active metabolite of the Selexipag.

It is an object of this invention to improve tolerability by reducing incidence of side effect associated with the Selexipag or its active metabolite. This is particularly important at high doses, for example at dose beyond 400 μg, at which the incidence of side effects can be relatively high. This object is effected by controlling the releases of the Selexipag or its active metabolite at a rate such that a mean or median Tmax (time to reach maximum plasma concentration) attained after administration of the pharmaceutical composition to mammal in fasting condition is at least greater than about 2.5 hours for the Selexipag, and/or at least greater than about 4 hours for the active metabolite of the Selexipag, thereby reducing the overall incidence as well as severity of the side effects and improving tolerability.

In a further embodiment, this invention provides a process for preparing controlled release pharmaceutical composition of the Selexipag or its active metabolite.

In a further embodiment, this invention provides a method for treating a Pulmonary Arterial Hypertension (PAH) with reduced incident of side effect, comprising administering the controlled release dosage form of the Selexipag or its active metabolite to a mammal, including a human patient, in need of such treatment which releases the Selexipag or its active metabolite according to the release rate described above or below.

In a further embodiment, this invention provides a controlled release pharmaceutical composition of Selexipag or its active metabolite that affords protection against alcohol dose dumping following oral administration with an alcoholic beverage, such that it provide a relative mean or median Tₘₐₓ of the selexipag in a range of about 25% to 150% of the relative mean or median Tₘₐₓ of the selexipag obtained following the oral administration of the same dose of the controlled release pharmaceutical composition of Selexipag or its active metabolite with a non-alcoholic beverage.

In a further embodiment, this invention provides a controlled release pharmaceutical composition of Selexipag or its active metabolite that affords protection against alcohol dose dumping following oral administration with an alcoholic beverage, such that it provide a relative mean or median Cₘₐₓ of the selexipag in a range of about 50% to 250%, preferably about 60% to 200%, more preferably about 75 to 150% and most preferably about 80% to 125% of the relative mean or median Cₘₐₓ of the selexipag obtained following the oral administration of the same dose of the controlled release pharmaceutical composition of Selexipag or its active metabolite with a non-alcoholic beverage.

The present invention also relates to a controlled release pharmaceutical composition of Selexipag or its active metabolite that provide bi-phasic release rate of selexipag or its active metabolite, wherein about 1 to 75% (preferably 5% to 70%, more preferably 10% to 60% and most preferably 15% to 50%) of the total amount of Selexipag or its active metabolite, respectively, contained in the pharmaceutical composition is released in the immediate release form and about 25 to 99% (preferably 30% to 95%, more preferably 40% to 90% and most preferably 50% to 85%) of the total amount of Selexipag or its active metabolite, respectively, contained in the pharmaceutical composition is released in the controlled release form.

### DETAILED DESCRIPTION OF THE INVENTION

The terms used in this specification generally have their ordinary meanings in the art, within the context of this invention and in the specific context where each term is used. Certain terms are discussed below, or elsewhere in the specification, to provide additional guidance to the practitioner in describing the compositions and methods of the invention and how to make and use them.

As used herein, the use of the word “a” or “an” when used in conjunction with the term “comprising” in the claims and/or the specification can mean “one,” but it is also consistent with the meaning of “one or more,” “at least one,” and “one or more than one.” Still further, the terms “having,” “including,” “containing”, “comprising” and “comprises” are interchangeable, and one of skill in the art is cognizant that these terms are open-ended terms.

The term “about” or “approximately” means within an acceptable error range for the particular value as determined by one of ordinary skill in the art. For example, “about” can mean a range of up to 20%, up to 15%, up to 10%, up to 5%, or up to 1% of a given value.

The present invention disclosed a controlled release formulation of Selexipag or its active metabolite that exhibits significant advantages over the convention immediate release formulation of the Selexipag (UPTRAVI®) of the prior arts. The innovative controlled release formulation of the Selexipag improve a tolerability by reducing an incidence of side effects compared to the conventional immediate release formulation of the Selexipag (UPTRAVI®) as disclosed in the prior arts.

In the present invention, the term “Side effects” means those physiological effects to various systems in the body such as cardiovascular, nervous, digestive, and the body as a whole, which cause discomfort to the individual subject, and which are the direct result of the ingestion of the Selexipag or its active metabolite, for example Headache.

In the present invention, the term “reducing incidence of side effects” refers to a reduced incidence of side effects in a patient population, and not to a total absence of side effects, when measured in a comparable population consuming a conventional immediate release dosage form of Selexipag (UPTRAVI®) suitable for twice daily administration. As it is well known to those skilled in the art, even placebo dosage forms of UPTRAVI© (placebo dosage form of UPTRAVI© means dosage form comprising all ingredient present in UPTRAVI® in similar amount except the Selexipag) produce some measurable incidence of side effects. Thus, an improved side effect profile must be interpreted in light of the relevant art.

In the present invention, the term “drug” or “drug substance” refers to Selexipag or its active metabolite.

The term “administration” or “ingestion” as used herein is essentially synonymous with “swallowing”.

The present invention is not limited to specific form of Selexipag or its active metabolite. Selexipag or its active metabolite can be employed in the pharmaceutical composition in any form including, but not limited to, in base form (as such), pharmaceutically acceptable form, any polymorphic form, anhydrous as well as hydrated form, solvates, co-crystal, crystalline or amorphous form, a single-component or multiple-component crystal, clathrate, etc. All such form of Selexipag or its active metabolite are within scope of this invention.

The pharmaceutical composition which constitute the subject matter of the invention are, as mentioned, controlled release formulations. The controlled release pharmaceutical composition of Selexipag or its active metabolite of the present invention can be administered to mammal by non-parenteral route, such as oral.

The term “controlled” is generic to “sustained”, “pulsatile”, “ascending” and “delayed”. The pharmaceutical composition of Selexipag or its active metabolite possess at least one of the following characteristics are not “controlled release”, and form no part of this invention:
1. provide mean or median Tmax less than 2 hours after administration to mammal in fasting condition, or
2. release more than 50% of their contained Selexipag or its active metabolite within 45 minutes or less when subjected to in-vitro dissolution testing at 37°±1° C. in 900 ml dissolution media having pH 6.8 in a USP Apparatus 2 (paddle) at 50 or 150 RPM.

For the purpose of this application, various embodiments of “controlled release dosage forms of Selexipag or its active metabolite” have been described as “sustained release” embodiments or “controlled release” embodiment, for ease of description. Without intending to be limiting, controlled release dosage forms of Selexipag or its active metabolite are those which slowly release Selexipag or its active metabolite; or release little or no Selexipag or its active metabolite for a predetermined time, then release Selexipag or its active metabolite quickly or in a sustained fashion; or release little or no Selexipag or its active metabolite in stomach but release Selexipag or its active metabolite quickly or in a sustained fashion in intestine. It will be appreciated by those skilled in the art that certain “sustained release” embodiments will also fall under the general rubric of “delayed release” or “pulsatile release” embodiments, and vice versa. For example, sustained release osmotic pump devices generally exhibit a “lag time” after ingestion, during which time the osmotic pressure in the device is increasing and during which time little or no drug is released. Thus, an osmotic pump device may be considered both a sustained release and a pulsatile release device.

In preferred embodiment, a controlled release pharmaceutical composition of a Selexipag or its active metabolite that improve tolerability by reducing incidence of side effect associated with the Selexipag or its active metabolite contained in the composition, wherein the composition comprising a release rate controlling ingredient in addition to the Selexipag or its active metabolite that control the releases of the Selexipag or its active metabolite at a rate such that a mean or median Tmax (time to reach maximum plasma concentration) attained after administration of the pharmaceutical composition to mammal in fasting condition is
a. at least greater than about 2.5 hours for the Selexipag, and/or
b. at least greater than about 4 hours for the active metabolite of the Selexipag, wherein the release rate controlling ingredient is present in the composition as
   a) matrixing agent in which the Selexipag or its active metabolite is embedded, or
   b) coating layer surrounding the Selexipag or its active metabolite, or
   c) coating layer surrounding an inert core comprising the Selexipag or its active metabolite, wherein the inert core further comprising at least one pharmaceutically acceptable ingredient in addition to the Selexipag or its active metabolite, or
   d) a combination of at least two steps from step a), step b) and step c).

In preferred embodiment, a controlled release pharmaceutical composition of a Selexipag or its active metabolite that improve tolerability by reducing incidence of side effect associated with the Selexipag or its active metabolite contained in the pharmaceutical composition comprising one or more pharmaceutically acceptable carriers in addition to the Selexipag or its active metabolite,
wherein a total amount of the pharmaceutically acceptable carriers contained in the pharmaceutical composition is not less than about 40% w/w of the total weight of the pharmaceutical composition,
wherein at least one pharmaceutically acceptable carrier is a release rate controlling ingredient and a ratio of the Selexipag or its active metabolite to the release rate controlling ingredient is from about 1:0.1 to about 1:1000, and
wherein the pharmaceutical composition releases the Selexipag or its active metabolite at a rate such that a mean or median Tmax (time to reach maximum plasma concentration) attained after administration of the pharmaceutical composition to mammal in fasting condition is
   a) at least greater than about 2.5 hours for the Selexipag, and/or
   b) at least greater than about 4 hours for the active metabolite of the Selexipag.

In preferred embodiment, a controlled release pharmaceutical dosage form of a Selexipag or its active metabolite that improve tolerability by reducing incidence of side effect associated with the Selexipag or its active metabolite contained in the dosage form comprising one or more pharmaceutically acceptable carriers in addition to the Selexipag or its active metabolite,
wherein a total amount of the pharmaceutically acceptable carriers contained in the dosage form is not less than about 40% w/w of the total weight of the dosage form, wherein at least one pharmaceutically acceptable carrier is a release rate controlling ingredient and a ratio of the Selexipag or its active metabolite to the release rate controlling ingredient is from 1:0.1 to 1:1000, and
wherein the dosage form having a release rate by weight of the Selexipag or its active metabolite in an aqueous medium is less than about 50% by weight of the Selexipag or its active metabolite release over the 45 minutes in the aqueous media, thereby a mean or median Tmax attained after administration of the dosage form to mammal in fasting condition is
   a) at least greater than about 2.5 hours for the Selexipag, and/or
   b) at least greater than about 4 hours for the active metabolite of the Selexipag,
   wherein the release rate is measured with USP apparatus 2 (paddle) at 50 RPM in 900 ml of an aqueous medium at a pH of 6.8±0.5 and temperature of 37°±1° C.,
wherein at least one side effect associated administration of the same amount of an immediate release dosage form of Selexipag or its active metabolite is reduced.

In preferred embodiment, a controlled release pharmaceutical composition of a Selexipag or its active metabolite that improve tolerability by reducing incidence of side effect associated with the Selexipag or its active metabolite contained in the composition, wherein the composition provide a release of Selexipag or its active metabolite at an ascending release rate over an extended period of time such that a mean or median Tmax (time to reach maximum plasma concentration) attained after administration of the pharmaceutical composition to mammal in fasting condition is
a. at least greater than about 2.5 hours for the Selexipag, and/or
b. at least greater than about 4 hours for the active metabolite of the Selexipag.

In preferred embodiment, a controlled release pharmaceutical dosage form that improve tolerability by reducing incidence of side effect associated with a Selexipag or its active metabolite contained in the dosage form comprising a release rate controlling ingredient in addition to the Selexipag or its active metabolite that govern a release of the Selexipag or its active metabolite from the dosage form, wherein the dosage form after administration to a mammal in fasting condition providing
a. a mean or median Tmax of at least greater than 2 hours for the Selexipag and/or at least greater than 3.5 hours for the active metabolite of the Selexipag, and/or
b. at least 20% lower Cmax for Selexipag or its active metabolite compared to Cmax obtained after administration of the same amount of an immediate release dosage form of Selexipag or its active metabolite in fasting condition,
wherein the release rate controlling ingredient is present in the composition as
a) matrixing agent in which the Selexipag or its active metabolite is embedded, or
b) coating layer surrounding the Selexipag or its active metabolite, or
c) coating layer surrounding an inert core comprising the Selexipag or its active metabolite, wherein the inert core further comprising at least one pharmaceutically acceptable ingredient in addition to the Selexipag or its active metabolite, or
d) a combination of at least two steps from step a), step b) and step c).

A controlled release pharmaceutical dosage form that improve tolerability by reducing incidence of side effect associated with a Selexipag or its active metabolite contained in the dosage form comprising a release rate controlling ingredient in addition to the Selexipag or its active metabolite that govern a release rate of the Selexipag or its active metabolite from the dosage form, wherein the release rate controlling ingredient allow to release Selexipag or its active metabolite from the dosage form in less than about 50% by weight of the Selexipag or its active metabolite contained in the dosage form over the 45 minutes in an aqueous medium, wherein the release rate is measured with USP apparatus 2 (paddle) at 50 or 150 RPM in 900 ml of an aqueous medium at a pH of 6.8±0.5 and temperature of 37°±1° C., wherein the release rate controlling ingredient is present in the composition as
a. matrixing agent in which the Selexipag or its active metabolite is embedded, or
b. coating layer surrounding the Selexipag or its active metabolite, or
c. coating layer surrounding an inert core comprising the Selexipag or its active metabolite, wherein the inert core further comprising at least one pharmaceutically acceptable ingredient in addition to the Selexipag or its active metabolite, or
d. a combination of at least two steps from step a), step b) and step c)

In a further embodiment, this invention provides a controlled release pharmaceutical composition that release the Selexipag or its active metabolite contained therein in an amount of less than about 60% (preferably less than about 50%) at 2 hours but more than about 80% at 8 hours when measured in 900 ml of phosphate buffer pH 6.8 using USP apparatus 2 (paddle) at 50 or 150 RPM and temperature of 37°±1° C., wherein the said controlled release pharmaceutical composition comprises at least one release rate controlling ingredient in amount of 2-98%, preferably in amount of 2-75%, more preferably in amount of 2.5 to 50% and most preferably in amount of 3 to 35% w/w of total weight of pharmaceutical composition.

In a further embodiment, this invention provides a controlled release pharmaceutical composition of Selexipag or its active metabolite that affords protection against alcohol dose dumping by releasing the Selexipag or its active metabolite in the amount of less than about 80%, preferably about 75%, more preferably about 70% and most preferably about 65% w/w of the total amount of the Selexipag or its active metabolite contained in the controlled release pharmaceutical composition at 2 hours when measured in 900 ml of 40% v/v ethanolic phosphate buffer pH 6.8 (360 mL of ethanol+540 mL of phosphate buffer pH 6.8) using USP apparatus 2 (paddle) at 50 RPM and temperature of 37°±1° C.

In a further embodiment, this invention provides a controlled release pharmaceutical composition of Selexipag or its active metabolite that provides protection against alcohol dose dumping. The effectiveness of this protection could also be demonstrated when a f2 value exceeding 50 is achieved, as determined by using a comparative dissolution profile of Selexipag or its active metabolite from the pharmaceutical composition in 900 ml of phosphate buffer pH 6.8 vs. 900 ml of 40% v/v ethanolic phosphate buffer pH 6.8 (comprising 360 ml of ethanol and 540 ml of phosphate buffer pH 6.8)

In a further embodiment, this invention provides a controlled release pharmaceutical composition of Selexipag or its active metabolite that affords protection against alcohol dose dumping following oral administration with an alcoholic beverage, such that it provide a relative mean or median Tₘₐₓ of the selexipag or its active metabolite in a range of about 25% to 150% of the relative mean or median Tₘₐₓ of the selexipag or its active metabolite, respectively, obtained following the oral administration of the same dose of the controlled release pharmaceutical composition of Selexipag or its active metabolite, respectively, with a non-alcoholic beverage.

In a further embodiment, this invention provides a controlled release pharmaceutical composition of Selexipag or its active metabolite that affords protection against alcohol dose dumping following oral administration with an alcoholic beverage, such that it provide a relative mean or median Cₘₐₓ of the selexipag or its active metabolite in a range of about 50% to 250%, preferably about 60% to 200%, more preferably about 75 to 150% and most preferably about 80% to 125% of the relative mean or median Cmax of the selexipag or its active metabolite, respectively, obtained following the oral administration of the same dose of the controlled release pharmaceutical composition of Selexipag or its active metabolite, respectively, with a non-alcoholic beverage.

The present invention also relates to a controlled release pharmaceutical composition of Selexipag or its active metabolite that provide bi-phasic release rate of selexipag or its active metabolite, wherein about 1 to 75% (preferably 5% to 70%, more preferably 10% to 60% and most preferably 15% to 50%) of the total amount of Selexipag or its active metabolite is released in immediate release form and about 25 to 99% (preferably 30% to 95%, more preferably 40% to 90% and most preferably 50% to 85%) of the total amount of Selexipag or its active metabolite is released in controlled release form.

In a further embodiment, this invention provides a method for treating a Pulmonary Arterial Hypertension (PAH) with reduced incident of side effect, comprising administering the controlled release dosage form of the Selexipag or its active metabolite to a mammal, including a human patient, in need of such treatment which releases the Selexipag or its active metabolite according to the release rate or pattern described as above.

In a further embodiment, this invention provides a method for treating a Pulmonary Arterial Hypertension (PAH) with reduced incident of side effect, comprising administering the controlled release pharmaceutical composition of Selexipag or its active metabolite that affords protection against alcohol dose dumping following oral administration with an alcoholic beverage, such that it provide a relative mean or median Tₘₐₓ of the selexipag or its active metabolite in a range of about 25% to 250%, preferably about 35% to 200%, more preferably about 35% to 150% of the relative mean or median Tₘₐₓ of the selexipag or its active metabolite, respectively, obtained following the oral administration of the same dose of the controlled release pharmaceutical composition of Selexipag or its active metabolite, respectively, with a non-alcoholic beverage.

In a further embodiment, this invention provides a method for treating a Pulmonary Arterial Hypertension (PAH) with reduced incident of side effect, comprising administering the controlled release pharmaceutical composition of Selexipag or its active metabolite that affords protection against alcohol dose dumping following oral administration with an alcoholic beverage provide a relative mean or median Cₘₐₓ of the selexipag or its active metabolite in a range of about 50% to 250%, preferably about 60% to 200%, more preferably about 75 to 150% and most preferably about 80% to 125% of the relative mean or median Cmax of the selexipag or its active metabolite obtained following the oral administration of the same dose of the controlled release pharmaceutical composition of Selexipag or its active metabolite, respectively, with a non-alcoholic beverage.

In a further embodiment, this invention provides a method for treating a Pulmonary Arterial Hypertension (PAH) with reduced incident of side effect, comprising administering the controlled release pharmaceutical composition of Selexipag or its active metabolite that affords protection against alcohol dose dumping following oral administration with an alcoholic beverage, wherein the said controlled release pharmaceutical composition of Selexipag or its active metabolite releases the Selexipag or its active metabolite in an amount of less than about 80% w/w, preferably about 75%, more preferably about 70% and most preferably about 65% of the total amount of the Selexipag or its active metabolite contained in the controlled release pharmaceutical composition at 2 hours when measured in 900 ml of 40% v/v ethanolic phosphate buffer pH 6.8 (360 mL of ethanol+540 mL of phosphate buffer pH 6.8) using USP apparatus 2 (paddle) at 50 RPM and temperature of 37°±1° C.

In a further embodiment, this invention provides a method for treating a Pulmonary Arterial Hypertension (PAH) with reduced incident of side effect, comprising administering the controlled release pharmaceutical composition of Selexipag or its active metabolite that affords protection against alcohol dose dumping following oral administration with an alcoholic beverage, wherein the said controlled release pharmaceutical composition release the Selexipag or its active metabolite contained therein in an amount of less than about 60% (preferably less than about 50%) at 2 hours but more than about 80% at 8 hours when measured in 900 ml of phosphate buffer pH 6.8 using USP apparatus 2 (paddle) at 50 or 150 RPM and temperature of 37°±1° C.

In further embodiment, this invention provides a method for treating a pulmonary arterial hypertension with reduced incident of side effect comprising administering a controlled release pharmaceutical composition of selexipag or its active metabolite, wherein the said controlled release pharmaceutical composition releases the selexipag or its active metabolite in an amount of less than about 75% of the total amount of the selexipag or its active metabolite contained therein at 2 hours when measured in 900 ml of 40% v/v ethanolic phosphate buffer pH 6.8 using USP apparatus 2 at 50 RPM and temperature of 37°±1° C., wherein the said controlled release pharmaceutical composition comprises at least one water soluble material, optionally in combination with water insoluble material, in an amount of 2% w/w to about 95% w/w (preferably from about 2.5% w/w to about 85% w/w, more preferably from about 3.5% w/w to about 75% w/w, most preferably from about 5% w/w to about 65% w/w) of the total weight of the pharmaceutical composition and is selected from the group of xanthan gum, acacia gum, diutan gum, tragacanth, gellan gum, guar gum, fenugreek gum, locust bean gum, pullulan, welan gum, polysaccharide, cellulose derivatives, and cellulose ether, wherein the said controlled release pharmaceutical composition comprises selexipag in an amount of 0.2 mg or more, wherein the said controlled release pharmaceutical composition release the selexipag or its active metabolite contained therein in an amount of less than about 60% at 2 hours but more than about 80% at 8 hours when measured in 900 ml of phosphate buffer pH 6.8 using USP apparatus 2 at 50 RPM and temperature of 37°±1° C.

In further embodiment, this invention provides a method for treating a pulmonary arterial hypertension with reduced incident of side effect using the controlled release pharmaceutical composition of selexipag or its active metabolite, wherein the controlled release pharmaceutical composition of selexipag or its active metabolite following oral administration with an alcoholic beverage reduces incidence of at least one side effect associated with the selexipag or its active metabolite compared to the immediate-release dosage form comprising the same amount of the selexipag or active metabolite thereof.

In preferred embodiment, for administration to the mammal by the non-parenteral route, the pharmaceutical composition can be in the form of a tablet, a capsule, a multiparticulates, suspension or a unit dose packet (sometimes refer to in the art as a “sachet”). The term “multiparticulate” is intended to embrace a pharmaceutical composition or dosage form comprising a multiplicity of particles whose totality represents the intended therapeutically useful dose of Selexipag or its active metabolite. The particles generally are of a diameter from about 50 microns to about 0.3 cm, with a preferred range of 100 M to 1.5 mm.

In preferred embodiment, the amount of Selexipag or its active metabolite contained within the dosage form is preferably at least 0.2 mg or more and most preferably at least 0.4 mg or more, 0.8 mg or more, 1.6 mg or more, 3.2 mg or more, 6.4 mg or more, 12.4 mg or more, 25 mg or more, but less than 500 mg. The term “dosage form” or “dosage unit” is used to refer to drug delivery system of the pharmaceutical composition comprising a predetermined quantity (therapeutically effective dose) of drug substance and suitable as unitary dosages for administration to human patients to attain the desired therapeutic effect.

The controlled release pharmaceutical composition of this invention, including those affords protection against alcohol dose dumping following oral administration with an alcoholic beverage, can be widely implemented, and for purposes of discussion, not limitation, the many embodiments hereunder can be grouped into classes according to design and principle of operation.

In preferred embodiment, a first class include matrix system, in which Selexipag or its active metabolite is embedded or suspended or dispersed within a matrix of controlled release ingredient which serve to retard the release of Selexipag or its active metabolite into an aqueous environment (i.e., the lumenal fluid of the GI tract, or dissolution media). When Selexipag or its active metabolite is dispersed in a matrix of this sort, release of the Selexipag or its active metabolite takes place principally from the surface of the matrix. Thus, the Selexipag or its active metabolite is released from the surface of the matrix after it diffuses through the matrix or when the surface of the matrix erodes, exposing the drug. In some embodiments, both mechanisms can operate simultaneously. The matrix systems may be large, i.e., tablet sized (about 1 cm), or small (<0.3 cm). The system may be unitary (e.g, a bolus), or may comprise a plurality of particles, referred to herein as a multiparticulate. A multiparticulate can be compressed with other pharmaceutically acceptable ingredient to form a tablet, or can be packed in capsule or sachet, or can be used to fabricate suspension (such as powder for suspension) or liquid formulation. A multiparticulate can have numerous formulation applications such as can be administered after mixing with food or juice.

The size of the matrix system can affect the rate of Selexipag or its active metabolite release, therefore, a large matrix system such as a tablet will, in general, have a different composition, i.e. comprising different controlled release ingredient or different amount of same controlled release ingredient, from a small one such as a multiparticulate. For purposes of further illustration, to obtain a sustained-release matrix in a particle of about 50 m in diameter, a matrix material of a polymer such as cellulose acetate, ethyl cellulose, wax or a similar material will likely be required. By contrast, in order to obtain sustained-release in a large (e.g., 1 cm) device, a material which is mostly liquid-like (e.g., a hydrogel, see below) will likely be required. For devices of an intermediate size, e.g., about 5 mm in diameter, a matrix material of intermediate characteristics can be employed. It is also noted that the effective diffusion coefficient of Selexipag or its active metabolite may be increased to the desired value by the addition of plasticizers, pores, or pore-inducing additives, as known in the art. Slowly-hydrating materials may also be used to give the desired intermediate diffusion rates. The multiplicity of variables affecting release of Selexipag or its active metabolite from matrix devices permits abundant flexibility in the design of devices of different materials, sizes, and release times. Certain examples of modifications of Selexipag or its active metabolite release profiles from the specific embodiments of the examples within the scope of this invention are disclosed below.

In one embodiment, a matrix multiparticulate, comprises a plurality of Selexipag or its active metabolite containing particles, each particle comprising a mixture of Selexipag or its active metabolite with one or more controlled release ingredient selected to form a matrix capable of limiting the dissolution rate of the Selexipag or its active metabolite into an aqueous medium. The matrix materials useful for this embodiment are generally water-insoluble materials or slowly water soluble material. If needed, the matrix materials may optionally be formulated with water-soluble materials which can be used as binders or as permeability-modifying agents.

A preferred process for manufacturing matrix multiparticulates is the extrusion/spheronization process. For this process, the Selexipag or its active metabolite is wet-massed with a binder, extruded through a perforated plate or die, and placed on a rotating disk. The extrudate ideally breaks into pieces which are rounded into spheres, spheroids, or rounded rods on the rotating plate.

A further preferred process for manufacturing matrix multiparticulates is the preparation of melt granules. In this process, a desired amount of Selexipag or its active metabolite is stirred with matrix material having low melting point at elevated temperature or above melting temperature of the matrix material to form a homogeneous mixture, cooled and then forced through a screen to form granules. Melt granules can be prepared by applying heat while mixing Selexipag or its active metabolite with matrix material having low melting point in granulator such as rapid mixture granulator.

A further preferred process for manufacturing matrix multiparticulates involves using an organic solvent to aid mixing of the Selexipag or its active metabolite with the matrix material. This technique can be used when it is desired to utilize a matrix material with an unsuitably high melting point that, if the material were employed in a molten state, would cause decomposition of the drug or of the matrix material, or would result in an unacceptable melt viscosity, thereby preventing mixing of Selexipag or its active metabolite with the matrix material. Selexipag or its active metabolite and matrix material may be combined with a modest amount of solvent to form a paste, and then forced through a screen to form granules from which the solvent is then removed. Alternatively, Selexipag or its active metabolite and matrix material may be combined with enough solvent to completely dissolve the matrix material and the resulting solution spray dried to form the particulate dosage form. This technique is preferred when the matrix material is a high molecular weight synthetic polymer such as a cellulose ether or cellulose ester or wax.

Once formed, Selexipag or its active metabolite matrix multiparticulates may be blended with compressible excipients and the blend compressed to form a tablet. Disintegrants are also usefully employed. Tablets prepared by this method disintegrate when placed in an aqueous medium, thereby exposing the multiparticulate matrix which releases Selexipag or its active metabolite. Selexipag or its active metabolite matrix multiparticulates may be filled in capsule or sachet or can be mixed with pharmaceutically acceptable additive to form a powder for suspension. Selexipag or its active metabolite matrix multiparticulates can be administered after sprinkling on food or juice.

A further embodiment of a matrix system has the form of a matrix tablet containing Selexipag or its active metabolite and an amount of matrixing material sufficient to provide a useful degree of control over the Selexipag or its active metabolite dissolution. The matrix tablet includes hydrophilic matrix, hydrophobic matrix, or any combination thereof. Hydrophilic polymer (Water soluble), hydrophobic material (water insoluble) or combination thereof are useful for forming the matrix. The Selexipag or its active metabolite is released by diffusion from the matrix, by erosion of the matrix or combination thereof. The Selexipag or its active metabolite dissolution rate from these matrix tablets may be controlled by the amount and/or the solubilisation rate of matrixing material employed. The dissolution rate may also be controlled by the use of water-soluble additives such as sugars, salts, or soluble polymers. In general, increasing the fraction of soluble material in the formulation increases the release rate.

The matrix systems as a class often exhibit non-constant release of the drug from the matrix. This result may be a consequence of the diffusive mechanism of drug release, and modifications to the geometry of the dosage form can be used to advantage to make the release rate of the drug more constant. In one embodiment, a Selexipag or its active metabolite matrix tablet is coated with an water insoluble coating in full or in part (on one or both tablet faces, or on the tablet radial surface). The water permeable coating optionally comprising pore former or flux enhancer such as water soluble additives like sugars, salts, or soluble polymers. The water insoluble coating may optionally comprising one or more passageways for drug transport from the composition to aqueous environment. A passageway for drug transport is produced by drilling a hole in coating, or removing one or more strip through the coating film, or cutting one or more slits through the coating film. In preferred embodiment, the water insoluble coating comprising at least one water insoluble material.

A further sustained release matrix system comprises Selexipag or its active metabolite dispersed in a hydrogel matrix. This embodiment differs from the hydrophilic matrix tablet discussed above in that the hydrogel of this embodiment is not a compressed tablet of erodible granular material, but rather a monolithic polymer network. As known in the art, a hydrogel is a water-swellable network polymer. Preferred materials for forming hydrogels include hydrophilic vinyl and acrylic polymers, polysaccharides such as calcium alginate, and poly(ethylene oxide). Especially preferred are poly(2-hydroxyethyl methacrylate), poly(acrylic acid), poly(methacrylic acid), poly(N-vinyl-2-pyrolidinone), poly(vinyl alcohol) and their copolymers with each other and with hydrophobic monomers such as methyl methacrylate, vinyl acetate, and the like. Also preferred are hydrophilic polyurethanes containing large poly(ethylene oxide) blocks. Other preferred materials include hydrogels comprising interpenetrating networks of polymers, which may be formed by addition or by condensation polymerization, the components of which may comprise hydrophilic and hydrophobic monomers such as those just enumerated.

In preferred embodiment, a second class of Selexipag or its active metabolite controlled release pharmaceutical composition of this invention includes membrane-moderated or reservoir systems. In this class, a reservoir (inert core) of Selexipag or its active metabolite is surrounded by a rate-limiting membrane (controlled release coating). These individual reservoir system dosage forms may be large, as in the case of a tablet or capsule containing a single large reservoir (inert core), or multiparticulate, as in the case of a plurality of reservoir (inert core) particles, each individually coated with the membrane. The coating can be non-porous, yet permeable to Selexipag or its active metabolite (for example Selexipag or its active metabolite may diffuse directly through the membrane), or it may be porous. As with other embodiments of this invention, the particular mechanism of transport is not believed to be critical.

Controlled release coatings as known in the art may be employed to fabricate the membrane, especially polymer coatings, such as a cellulose ester or ether, an acrylic polymer, vinyl acetate (such as polyvinyl acetate) or its copolymer or a mixture of polymers, on a reservoir of Selexipag or its active metabolite. The polymer may be applied as a solution in an organic solvent or an aqueous solvent or as an aqueous dispersion or latex. The coating operation may be conducted in standard equipment such as a fluid bed coater, a Wurster coater, or a rotary bed coater or conventional coater.

If desired, the permeability of the coating may be adjusted by blending of two or more materials. A particularly useful process for tailoring the porosity of the coating comprises adding a pre-determined amount of a finely-divided water-soluble material, such as sugars or salts or water-soluble polymers to a solution or dispersion (e.g., an aqueous latex) of the membrane-forming polymer to be used. When the dosage form is ingested into the aqueous medium of the GI tract, these water soluble membrane additives are leached out of the membrane, leaving pores which facilitate release of the drug. The membrane coating can also be modified by the addition of plasticizers, as known in the art.

A particularly useful variation of the process for applying a membrane coating comprises dissolving the coating polymer in a solvent or mixture of solvents chosen such that as the coating dries, a phase inversion takes place in the applied coating solution, resulting in a membrane with a porous structure. Numerous examples of this type of coating system are given in European Patent Specification 0357369 B1, published Mar. 7, 1990, herein incorporated by reference. In general, a support for mechanically strengthening the membrane is not required.

The morphology of the membrane is not of critical importance so long as the permeability characteristics enumerated herein are met. The membrane can be amorphous or crystalline. It can have any category of morphology produced by any particular process and can be, for example, an interfacially-polymerized membrane (which comprises a thin rate-limiting skin on a porous support), drug permeable membrane, asymmetric membrane, a porous hydrophilic membrane, a porous hydrophobic membrane, a hydrogel membrane, an ionic membrane, and other such materials which are characterized by controlled permeability to Selexipag or its active metabolite.

A single larger reservoir such as tablet or capsule can be manufactured by the process known in this art using, in addition of the drug substance, one or more pharmaceutical acceptable material which include, but not limited to, filler, binder, osmotic agents, hydrophilic, swellable or hydrogel polymer, lubricant, glidant, surfactant, and like.

A preferred embodiment of the class of reservoir systems comprises a multiparticulate, wherein each particle is coated with a polymer designed to yield controlled release of Selexipag or its active metabolite. The multiparticulate particles each comprise Selexipag or its active metabolite and one or more excipients as needed for fabrication and performance including ion-exchange resin to form drug-ion-exchange resin complex. The size of individual particles is generally between about 50 m and about 3 mm, although beads of a size outside this range may also be useful.

Reservoir system Selexipag or its active metabolite multiparticulates may be prepared using techniques known to those skilled in the art, including, but not limited to, the techniques of extrusion and spheronization, wet granulation, fluid bed granulation, rotary bed granulation and forming complexation of drug with ion-exchange resin (drug-ion exchange resin complex). In addition, the beads may also be prepared by building the Selexipag or its active metabolite composition (drug plus excipients) up on a seed core (such as a non-pareil seed) by a drug-layering technique such as powder coating or by applying the Selexipag or its active metabolite composition by spraying a solution or dispersion of Selexipag or its active metabolite in an appropriate binder solution onto seed cores (such as sugar sphere, microcrystalline cellulose sphere, etc) in a fluidized bed such as a Wurster coater or a rotary processor. Another method for manufacturing the multiparticulate cores of this embodiment is the extrusion and spheronization process, as previously discussed for matrix multiparticulates.

A controlled release coating as known in the art, especially polymer coatings, may be employed to fabricate the membrane, as previously discussed, for reservoir systems. Suitable and preferred polymer coating materials, equipment, and coating methods also include those previously discussed.

The rate of Selexipag or its active metabolite release from the coated multiparticulates can also be controlled by factors such as the composition and binder content of the drug-containing core (reservoir), the thickness and permeability of the coating, and the surface-to-volume ratio of the multiparticulates. It will be appreciated by those skilled in the art that increasing the thickness of the coating will decrease the release rate, whereas increasing the permeability of the coating or the surface-to-volume ratio of the multiparticulates will increase the release rate. If desired, the permeability of the coating may be adjusted by blending of two or more materials. A useful series of coatings comprises mixtures of water-insoluble and water-soluble polymers, for example, ethylcellulose and hydroxypropyl methylcellulose, respectively. A particularly useful modification to the coating is the addition of finely-divided water-soluble material, such as sugars or salts. When placed in an aqueous medium, these water soluble membrane additives are leached out of the membrane, leaving pores which facilitate delivery of the drug. The membrane coating may also be modified by the addition of plasticizers, as is known to those skilled in the art. A particularly useful variation of the membrane coating utilizes a mixture of solvents chosen such that as the coating dries, a phase inversion takes place in the applied coating solution, resulting in a membrane with a porous structure.

In preferred embodiment, a third class of Selexipag or its active metabolite controlled release pharmaceutical composition includes the osmotic delivery devices or “osmotic pumps” as they are known in the art. Osmotic pumps comprise a core containing an osmotically effective composition surrounded by a semipermeable membrane. The term “semipermeable” in this context means that water can pass through the membrane, but solutes dissolved in water cannot. In use, when placed in an aqueous environment, the device imbibes water due to the osmotic activity of the core composition. Owing to the semipermeable nature of the surrounding membrane, the contents of the device (including the drug and any excipients) cannot pass through the non-porous regions of the membrane and are driven by osmotic pressure to leave the device through an opening or passageway pre-manufactured into the dosage form or, alternatively, formed in situ in the GI tract as by the bursting of intentionally-incorporated weak points in the coating under the influence of osmotic pressure. The osmotically effective composition includes water-soluble species, which generate a colloidal osmotic pressure, and water-swellable polymers.

Materials useful for forming the semipermeable membrane include polyamides, polyesters, and cellulose derivatives. Preferred are cellulose ethers and esters. Especially preferred are cellulose acetate, cellulose acetate butyrate, and ethyl cellulose. Especially useful materials include those which spontaneously form one or more exit passageways, either during manufacturing or when placed in an environment of use. These preferred materials comprise porous polymers, the pores of which are formed by phase inversion during manufacturing, as described above, or by dissolution of a water-soluble component present in the membrane.

A preferred embodiment of this class of osmotic delivery devices consists of a coated mono-layer, bi-layer or tri-layer tablet. The coated monolayer tablet comprising a single layer tablet core containing the Selexipag or its active metabolite composition and a semipermeable coating surrounding the core tablet contains one or more exit passageways. The coated bilayer tablet comprising (1) a tablet core consists of two layers: one layer containing the Selexipag or its active metabolite composition and another layer consisting of an expandable hydrogel (fluid expandable polymer), with or without additional osmotic agents; and (2) a semipermeable coating surrounding the core tablet contains one or more exit passageways in communication with the Selexipag or its active metabolite containing layer for delivering the drug composition. The coated trilayer tablet comprising (1) a tablet core consists of three layers: first layer comprising the Selexipag or its active metabolite composition, second layer comprising the Selexipag or its active metabolite composition and another layer consisting of an expandable hydrogel (fluid expandable polymer), with or without additional osmotic agents, wherein % weight of the Selexipag or its active metabolite in the second layer with respect to total weight of the second layer is higher than % weight of the Selexipag or its active metabolite in the first layer with respect to total weight of the first layer, and (2) a semipermeable coating surrounding the core tablet contains one or more exit passageways in communication with the first Selexipag or its active metabolite containing layer for delivering the drug composition, wherein the trilayer coated tablet deliver the Selexipag or its active metabolite at ascending rate over the extended period of time, for at least about 4 hours or more. The layer of Selexipag or its active metabolite composition comprises, in addition of the drug substance, one or more pharmaceutical acceptable material which include, but not limited to, filler, binder, osmotic agents, osmo-polymer (fluid expandable polymer such as polyethylene oxide, hydroxyethyl cellulose and like), hydrophilic or swellable polymer, lubricant, glidant, surfactant, and like.

The rate of Selexipag or its active metabolite delivery is controlled by such factors as the permeability and thickness of the coating, the water activity of the hydrogel layer, and the surface area of the device. Those skilled in the art will appreciate that increasing the thickness of the coating will reduce the release rate, whereas increasing the permeability of the coating or the water activity of the hydrogel layer or the surface area of the device will increase the release rate.

The exit passageway must be located on the side of the tablet containing the Selexipag or its active metabolite composition. There may be more than one such exit passageway. The exit passageway may be produced by mechanical or by laser drilling, or by creating a difficult-to-coat region on the tablet by use of special tooling during tablet compression.

The osmotic pump also includes osmotic capsule. The method of manufacturing osmotic capsule as well as osmotic tablets is well known in the art, are described in detailed in many patents issued to Alza Corp as well as in “Osmotic drug delivery: a review of the patent literature”, Journal of Controlled Release 35 (1995) 1-21.

In preferred embodiment, a forth class Selexipag or its active metabolite controlled release pharmaceutical composition comprising pH sensitive coat surrounding any one of the first class, the second class and third class controlled release pharmaceutical composition of Selexipag or its active metabolite discussed aforesaid, preferably the coat of a pH sensitive polymer which is substantially insoluble and impermeable at the pH of the stomach, and which is more soluble and permeable at the pH of the small intestine. Preferably, the coating polymer is substantially insoluble and impermeable at pH<5.0, and water soluble at pH>5.0. The pH-sensitive polymers which are relatively insoluble and impermeable at the pH of the stomach, but which are more soluble and permeable at the pH of the small intestine and colon include polyacrylamides, phthalate derivatives such as acid phthalates of carbohydrates, amylose acetate phthalate, cellulose acetate phthalate, other cellulose ester phthalates, cellulose ether phthalates, hydroxypropylcellulose phthalate, hydroxypropylethylcellulose phthalate, hydroxypropylmethylcellulose phthalate, methylcellulose phthalate, polyvinyl acetate phthalate, polyvinyl acetate hydrogen phthalate, sodium cellulose acetate phthalate, starch acid phthalate, styrene-maleic acid dibutyl phthalate copolymer, styrene-maleic acid polyvinylacetate phthalate copolymer, styrene and maleic acid copolymers, polyacrylic acid derivatives such as acrylic acid and acrylic ester copolymers, polymethacrylic acid and esters thereof, poly acrylic methacrylic acid copolymers, shellac, and vinyl acetate and crotonic acid copolymers. The pH sensitive coat comprising an amount of polymer from about 5 to about 50% of total weight of the pharmaceutical composition.

In preferred embodiment, a fifth class Selexipag or its active metabolite controlled release pharmaceutical composition comprising pH sensitive coat surrounding a reservoir of Selexipag or its active metabolite. In this class, a reservoir of Selexipag or its active metabolite is surrounded by the pH sensitive coat. These individual reservoir system dosage forms may be large, as in the case of a tablet or capsule containing a single large reservoir, or multiparticulate, as in the case of a plurality of reservoir particles, each individually coated with the pH sensitive polymer.

Method of manufacturing the reservoir of Selexipag or its active metabolite are described aforesaid in the second class of Selexipag or its active metabolite controlled release pharmaceutical composition.

In preferred embodiment, the first class, the second class, the third class, the fourth class and the fifth class controlled release pharmaceutical composition of Selexipag or its active metabolite described above further comprising immediate release dose of Selexipag or its active metabolite to provide an initial rapid onset of the drug action. The immediate release dose of Selexipag or its active metabolite can be provided in the pharmaceutical composition in form of coating layer or in form of separate layer in multilayer tablet or in form of granules.

In a further embodiment, this invention provides a controlled release pharmaceutical composition of Selexipag or its active metabolite that affords protection against alcohol dose dumping following oral administration with an alcoholic beverage, wherein the said controlled release pharmaceutical composition comprising a mixture of water insoluble material and water soluble material (such as hydrophilic polymer, swellable polymer, hydrogel polymer and water soluble polymer) in the ratio from 50:50 to 97:3, preferably 60:40 to 96:4, more preferably 70:30 to 96:4 and most preferably 80:20 to 96:4, respectively, wherein the said mixture of water insoluble material and water soluble material is included in the composition in coating form surrounding the core of selexipag or selexipag is embedded within the said mixture of water insoluble material and water soluble material. In certain embodiments, the preferable water-soluble material are selected from the group of gum such as xanthan gum, acacia gum, diutan gum, tragacanth, gellan gum, guar gum, fenugreek gum, locust bean gum, pullulan, welan gum; polysaccharide; cellulose derivatives; and cellulose ether.

In the present invention, the terms “material”, “excipient”, “ingredient” and “component” are interchangeable, and one of skill in the art is cognizant that these terms are open-ended terms.

According to present invention, a controlled release materials includes water-insoluble materials, hydrophilic polymer, swellable polymer, hydrogel polymer, water soluble polymer and pH sensitive polymer. In the present invention, the term “controlled release material” or “release rate controlling ingredient” or “release retardant” are interchangeable. The term “controlled release material” refers to any material that control or sustained the release rate of Selexipag or its active metabolite from a dosage form. In preferred embodiment, the dosage form or pharmaceutical composition comprising controlled release material in about 0.01% w/w to about 99.9% w/w, preferably about 1% w/w to about 95% w/w, more preferably about 2% w/w to about 93% w/w, most preferably about 10% w/w to about 85% w/w or particularly preferably about 15% w/w to about 75% w/w of total weight of the dosage form or total weight of the pharmaceutical composition.

In the present invention, the term “water insoluble material” or “hydrophobic material” refers to a component which is insoluble in water. A water insoluble material includes wax and water insoluble polymer. Suitable water insoluble ingredient includes natural, synthetic or semi synthetic ingredient. Natural, synthetic or semi synthetic water insoluble ingredient include, but are not to be limited, cellulose derivatives include cellulose acetate, cellulose acetate butyrate, cellulose triacetate, microcrystalline cellulose, ethyl cellulose; wax include microcrystalline wax, beeswax, glycowax, castor wax, carnauba wax, glycerol monostearate, glycerol palmitostearate; oil include hydrogenated vegetable oil, hydrogenated castor oil, vegetable oil, stearyl alcohol, acetylated hydrogenated soybean oil glycerides, castor oil; glycerol behenic acid ester, glyceryl monooleate, glyceryl monostearate, propylene glycol monostearate, cetyl alcohol, natural and synthetic glycerides, fatty acids, fatty alcohol, lipid, methacrylic acid derivatives such as polymethacrylate and its copolymer (such as polymer under brand name Eudragit®), polyvinyl acetate, copolymers of vinyl pyrrolidone and vinyl acetate; vinyl acetate and copolymer thereof, ethyl vinyl acetate, modified starch like pregelatinised starch, polylactic acid or polyglycolic acid and copolymers thereof, methacrylates, cocoa butter, macrogol Stearate, diethylene glycol monostearate, polyoxyethylene 50 stearate, and mixtures thereof. In preferred embodiment, the dosage form or pharmaceutical composition comprising water insoluble material or hydrophobic material in about 0.01% w/w to about 99.9% w/w, preferably about 1% w/w to about 95% w/w, more preferably about 2% w/w to about 93% w/w, most preferably about 10% w/w to about 85% w/w or particularly preferably about 15% w/w to about 75% w/w of total weight of the dosage form or total weight of the pharmaceutical composition.

A suitable hydrophilic polymer, swellable polymer, hydrogel polymer and water soluble polymer consisting of natural, semi-synthetic or synthetic ingredient which includes, but not limited to, gum such as xanthan gum, acacia gum, diutan gum, tragacanth, gellan gum, guar gum, fenugreek gum, locust bean gum, pullulan, welan gum; polysaccharide; polymer includes, but not limited to, cellulose derivatives such as carboxymethyl cellulose, cellulose ether (such as hydroxyethyl cellulose, hydroxypropylmethyl cellulose, hydroxypropyl cellulose, etc), polyalkylene oxide and its co-polymer such as polyethylene oxide, copolymer of ethylene oxide-propylene oxide, ploycarboxylic acid such as polyacrylic acid, polyolefinic alcohol (such as polyvinyl alcohol), or a polyvinyl lactam such as, e.g., polyvinylpyrrolidone, polyvinyl caprolactam, alginic acid and its derivative, methacrylic acid and its copolymer (such as polymer under brand name Eudragit®), polyacrylic acid and copolymer thereof (such as carbomer); protein or polypeptide such as gelatin, albumin, polylysine, soy protein; starch or its derivative; and like. Polysaccharides are polymeric carbohydrate molecules composed of long chains of monosaccharide units bound together by glycosidic linkages and on hydrolysis give the constituent monosaccharides or oligosaccharides. They range in structure from linear to highly branched. Examples include storage polysaccharides such as starch and glycogen, and structural polysaccharides such as cellulose and chitin. In certain embodiment, polymer, gum, polysaccharide and starch can be used as binder, pore former, flux enhancer and osmo-polymer (osmotic agent) like. In preferred embodiment, the dosage form or pharmaceutical composition comprising swellable polymer, hydrogel polymer or water soluble polymer in about 0.01% w/w to about 99.9% w/w, preferably about 1% w/w to about 95% w/w, more preferably about 2% w/w to about 93% w/w, most preferably about 10% w/w to about 85% w/w or particularly preferably about 15% w/w to about 75% w/w of total weight of the dosage form or total weight of the pharmaceutical composition.

In the present invention, the term “hydrophilic material” refers to a component which is soluble in water. The hydrophilic material includes, but not limited to, hydrophilic polymer, swellable polymer and hydrogel polymer, salt, sugar, acid, base and like.

The controlled release pharmaceutical composition further comprising one or more pharmaceutically acceptable additive depending on its functionality in the pharmaceutical composition which includes, but not limited to, filler, diluent, disintegrant, anti-tacking agent, binder, glidant, surfactant, wetting agent, lubricant, anti-oxidant, plasticizer, sweetener, coloring agent, sugar, salt, acid and osmotic agent.

Fillers or diluents such as cellulose derivatives, mono, di or tri basic calcium phosphate, sugar, carbohydrate, starch derivatives, acid or base, and like. Filler or diluent refers to material that is used to increase the bulk volume of a dosage form and/or to improve content uniformity of a dosage form. Filler or diluent does not inter

Disintegrant may be highly/rapidly swellable, moderately swellable or slowly swellable such as vinylpyrrolidone polymers such as crospovidon, cellulose and cellulose derivatives, sodium starch glycolate, starch and starch derivatives, resins, and like

Suitable anti-tacking agent is selected from the group consisting of, but are not limited to, stearates, stearic acid, vegetable oil, waxes, a blend of magnesium stearate and sodium lauryl sulfate, boric acid, surfactants, sodium benzoate, sodium acetate, sodium chloride, DL-Leucine, polyethylene glycol, sodium oleate, sodium lauryl sulfate, magnesium lauryl sulfate, talc, corn starch, amorphous silicon dioxide, syloid, metallic stearates, Vitamin E, Vitamin E TPGS, silica and combinations thereof.

Suitable binder include, but are not to be limited, cellulose derivatives include, but are not limited to be, methyl cellulose, hydroxypropyl methylcellulose, hydroxyethyl cellulose, hydroxypropyl cellulose, hydroxyethyl methylcellulose, ethylhydroxyethylcellulose, ethylmethylcellulose, hydroxymethylcellulose, hydroxymethylpropylcellulose, sodium carboxymethylcellulose, polyacrylamide derivatives, methacrylic acid derivatives, vinyl pyrrolidone polymers such as polyvinylpyrrolidone, starch derivative, polyalkylene oxide and copolymer thereof, alkylene oxide homopolymers, gums of plant, animal, mineral or synthetic origin, polyacrylic acid and copolymer thereof, polyvinyl alcohols, polyethylene glycol, poloxamer, and mixtures thereof.

Suitable glidants and lubricants may be incorporated such as stearic acid, metallic stearates, talc, waxes, and glycerides with high melting temperatures, colloidal silica, sodium stearyl fumarate, polyethyleneglycols, and alkyl sulphates.

Suitable plasticizers includes, but are not limited to, triacetin, triethyl acetate, acetylated monoglyceride, olive oil, acetyl tributyl citrate, acetyl triethyl citrate, glycerin, sorbitol, polyethylene glycol, polypropyleneglycol and like.

Stabilizer, such as anti-oxidant, to inhibit or retard oxidative drug decomposition during storage of the pharmaceutical composition.

Natural or synthetic sweeteners include, but note limited to, mannitol, sorbitol, saccharose, saccharine, aspartame, acelsulphame K, or cyclamate.

Preferred coloring agent includes D&C dye/lake, FD&C dye, an FD&C lake, caramel, ferric oxide, a natural coloring agent, and a combination thereof. The amount of coloring agent used will vary as desired. Preferably the composition is non-toxic, edible, stable in light and air and free of potential hazards to human health.

Preferred sugars include dextrose, glucose, arabinose, ribose, arabinose, xylose, lyxose, xylol, allose, altrose, inositol, glucose, sorbitol, mannose, gulose, glycerol, idose, galactose, talose, trehalose, mannitol, erythritol, ribitol, xylitol, maltitol, isomalt, lactitol, sucrose, raffinose, maltose, fructose, lactose, dextrin, dextran, amylase and xylan.

Water soluble salts include sodium chloride, potassium chloride, calcium chloride or magnesium chloride, lithium chloride, lithium, sodium or potassium hydrogen phosphate, lithium, sodium or potassium dihydrogen phosphate, salts of organic acids such as sodium or potassium acetate, sodium bicarbonate, magnesium succinate, sodium benzoate, sodium citrate or sodium ascorbate.

Preferred acids include ascorbic acid, 2-benzene carboxylic acid, benzoic acid, fumaric acid, citric acid, maleic acid, serbacic acid, sorbic acid, edipic acid, edetic acid, glutamic acid, toluene sulfonic acid, water-soluble amino acids such as glycine, leucine, alanine, or methionine and tartaric acid; and like.

Osmotic agent includes sugar and salts as described above.

Surfactants and Wetting agents include, but are not limited to heptadecaethylene oxycetanol, lecithins, sorbitol monooleate, polyoxyethylene sorbitol monooleate, polyoxyethylene stearate, polyoxyethylen sorbitan monolaurate, benzalkonium chloride, nonoxynol 10, oxtoxynol 9, polysorbates for example 20, 40, 60 or 80, sorbitan mono-palmitate, sodium salts of fatty alcohol-sulfates such as sodium lauryl sulfate, sodium dodecylsulfate, sodium salts of sulfosuccinates such as sodium dioctylsulfosuccinate, partially esters of fatty acids with alcohols such as glycerine monostearate, partially esters of fatty acids with sorbitans such as sorbitan monolaurate, partially esters of fatty acids with polyhydroxyethylene sorbitans such as polyethyleneglycol sorbitan monolaurate, -monostearate or -monooleate, ethers of fatty alcohols with polyhydroxyethylene, esters of fatty acids with polyhydroxyethylene, copolymers of ethylenoxide and propylenoxide (Pluronic®) and ethoxylated triglycerides.

Functionality of pharmaceutically acceptable additive and tentative range for concentration to be used in the pharmaceutical dosage form according to the functionality also described in Handbook of Pharmaceutical Excipients, 6^{th} Edition, Edited by Raymond C Rowe, Paul J Sheskey and Marian E Quinn.

The above listed ingredients are for information only, which does not limit the scope of the present invention. Any ingredient which is used in the pharmaceutical composition that fulfil the subject matter of the present invention are within the scope of the present invention.

The present invention is not limited to art of fabricating controlled release dosage form of Selexipag or its active metabolite, in other word, an ordinary skill in this art can also manufactured controlled release dosage form of Selexipag or its active metabolite by any other method than described above.

### EXAMPLES

The invention will now be illustrated by the following examples which are not to be taken as limiting. In general, the examples demonstrate the preparation of controlled-release pharmaceutical composition of Selexipag or its active metabolite within the scope of this invention.

### Example 1: Matrix Formulation (Tablet Form)

| Sr. No. | Ingredient Name | % w/w |
|---|---|---|
| 1 | Selexipag | 0.67 |
| 2 | Mannitol | 66.66 |
| 3 | Hydroxypropylmethyl Cellulose (HPMC) | 30.17 |
| 4 | Colloidal Silicon Dioxide | 1.50 |
| 5 | Magnesium Stearate | 1.00 |

Manufacturing Procedure:
1. Co-sift Selexipag with mannitol through appropriate screen.
2. Sift Hydroxypropylmethyl cellulose, colloidal silicon dioxide and magnesium stearate separately through appropriate screen.
3. Blend the step (1) Selexipag and mannitol with the step (2) HPMC and colloidal silicon dioxide for suitable time period.
4. Blend the step (2) magnesium stearate with the step (3) material for suitable period of time.
5. Compress the tablets using the step (4) material on compression machine using appropriate tooling (punches).

### Example 2: Coated Reservoir Formulation (Multiparticulate Form)

| | | |
|---|---|---|
| Sr. No. | Ingredient Name | % w/w |

Reservoir Components (Sustained Release)

| | | |
|---|---|---|
| 1 | Selexipag | 0.67 |
| 2 | Mannitol | 41.28 |
| 3 | Hydroxypropylmethyl Cellulose (HPMC) | 40.00 |
| 4 | Colloidal Silicon Dioxide | 0.83 |
| 5 | Magnesium Stearate | 0.56 |

Coating Components (Sustained Release)

| | | |
|---|---|---|
| 6 | Ethyl Cellulose | 6.67 |
| 7 | Hydroxypropylmethyl Cellulose (HPMC) | 4.44 |

Immediate Release Drug Coat

| | | |
|---|---|---|
| 8 | Selexipag | 0.44 |
| 9 | Hydroxypropylmethyl Cellulose (HPMC) | 3.72 |
| 10 | Polyethylene Glycol (PEG) | 1.39 |

Manufacturing Procedure:
A. Preparation of Resevoir Core
1. Co-sift Selexipag with mannitol through appropriate screen.
2. Sift Hydroxypropylmethyl cellulose, colloidal silicon dioxide and magnesium stearate separately through appropriate screen.
3. Blend the step (1) Selexipag and mannitol with the step (2) HPMC and colloidal silicon dioxide for suitable time period.
4. Blend the step (2) magnesium stearate with the step (3) material for suitable period of time.
5. Compress the mini-tablets using the step (4) material on compression machine using 2 mm tooling (punches).
B. Sustained Release Coat
6. Preparation of Coating Solution: Add one by one coating components, ethyl cellulose following HPMC, in mixture of dichloromethane and isopropyl alcohol (1:1) and stir it till get clear solution.
7. Coat the step (5) minitablets in coating machine using the step (6) coating solution.
C. Immediate Release Drug Over Coat
8. Preparation of Coating Solution: Add one by one coating components, Selexipag followed by PEG followed by HPMC, in water and stir it for at least 60 minutes.
9. Coat the step (7) minitablets in coating machine using the step (8) coating solution.

### Example 3: Osmotic Formulation (For Ascending Drug Release Profile)

| | | |
|---|---|---|
| Sr. No. | Ingredient Name | % w/w |

Trilayer Tablet Core (First Layer)

| | | |
|---|---|---|
| 1 | Selexipag | 0.59 |
| 2 | Polyethylene Oxide (Polyox WSR N80) (PEO) | 25.95 |
| 3 | Povidone (PVP) | 2.44 |
| 4 | Ferric Oxide Red (color) | 0.05 |
| 5 | Magnesium Stearate | 0.24 |

Trilayer Tablet Core (Second Layer)

| | | |
|---|---|---|
| 6 | Selexipag | 0.78 |
| 7 | Polyethylene Oxide (Polyox WSR N80) (PEO) | 16.98 |
| 8 | Povidone (PVP) | 1.59 |
| 9 | Brilliant Blue (color) | 0.05 |
| 10 | Magnesium Stearate | 0.12 |

Trilayer Tablet Core (Third Layer)

| | | |
|---|---|---|
| 11 | Polyethylene Oxide (Polyox WSR N303) (PEO) | 26.83 |
| 12 | Sodium Chloride | 7.80 |
| 13 | Povidone (PVP) | 3.90 |
| 14 | Magnesium Stearate | 0.49 |

Semipermeable Membrane Coat

| | | |
|---|---|---|
| 15 | Cellulose Acetate | 7.90 |
| 16 | Polyethylene Glycol | 0.88 |

Immediate Release Drug Coat

| | | |
|---|---|---|
| 17 | Selexipag | 0.20 |
| 18 | Hydroxypropylmethyl Cellulose (HPMC) | 2.44 |
| 19 | Polyethylene Glycol (PEG) | 0.78 |

Manufacturing Procedure:
A. Preparation of First Layer and Second Layer granules
1. Preparation of Binder Solution: Add PVP in Isopropyl Alcohol (95%) and stir it till get clear solution.
2. Co-sift Selexipag, PEO and color through appropriate screen. Sift magnesium stearate through appropriate screen.
3. Transfer the step (2) Selexipag, PEO and color in rapid mixture granulator and mix it for 15 minutes.
4. Perform granulation of the step (3) material using the step (1) binder solution.
5. Dry the step (4) wet granules in dryer till desired LOD is achieve.
6. Sift the step (5) granules through appropriate screen and mill the oversize granules, if any, retain on the screen in appropriate mill using appropriate milling screen.
7. Blend the step (6) granules with the step (2) magnesium stearate in blender for appropriate time at appropriate blending speed.
B. Preparation of Third Layer granules
8. Preparation of Binder Solution: Add PVP in Isopropyl Alcohol (95%) and stir it till get clear solution.
9. Sift PEO and sodium chloride through appropriate screen. Sift magnesium stearate through appropriate screen.
10. Transfer the step (9) PEO and sodium chloride in rapid mixture granulator and dry mix it for 10 minutes.
11. Perform granulation of the step (10) material using the step (8) binder solution.
12. Dry the step (11) wet granules in dryer.
13. Sift the step (12) granules through appropriate screen and mill the oversize granules, if any, retain on the screen in appropriate mill using appropriate milling screen.
14. Blend the step (13) granules with the step (9) magnesium stearate in blender for appropriate time at appropriate blending speed.
C. Trilayer Tablet Preparation
15. Compress the Trilayer tablet on compression machine with appropriate hardness by first filling the first layer granules followed by the second layer granules followed by third layer granules.
D. Semipermeable Membrane Coating
16. Preparation of coating solution: Add polyethylene glycol followed by cellulose acetate in mixture of Acetone:Water (99:1) and stir it till get clear solution.
17. Coat the step (15) Trilayer tablet in coater using step (16) coating solution using appropriate coating parameters.
E. Drilling
18. Drill the step (17) tablets on the first layer side (red color layer).
F. Immediate Release Drug Over Coat
19. Preparation of Coating Solution: Add one by one coating components, Selexipag followed by PEG followed by HPMC, in water and stir it for at least 60 minutes.
20. Coat the step (18) tablets in coater using the step (19) coating solution using appropriate coating parameters.

### Example 4: Coated Reservoir Formulation (Multiparticulate Form) that Affords Protection Against Alcohol Dose Dumping

| | |
|---|---|
| Example 4A | Example 4B |

| | | |
|---|---|---|
| Sr. No. | Ingredient Name | Mg/Capsule |

Reservoir Components (Sustained Release)

| | | |
|---|---|---|
| 1 | MCC Spheres | 105.00 |
| 2 | Selexipag | 1.20 |
| 3 | Mannitol | 10.40 |
| 4 | Hydroxypropylmethyl Cellulose (HPMC) | 13.40 |

Coating Components (Sustained Release)

| | | | |
|---|---|---|---|
| 5 | Ethyl Cellulose (from Aqua Coat ECD 30) | 13.81 | 13.81 |
| 6 | Guar Gum | 2.44 | — |
| 7 | Hypromellose | — | 2.44 |

Immediate Release Drug Coat

| | | |
|---|---|---|
| 8 | Selexipag | 0.40 |
| 9 | Mannitol | 3.50 |
| 10 | Hydroxypropylmethyl | 4.85 |
| | Cellulose (HPMC) | |
| | Total | 155.00 |

Manufacturing Procedure:
1. Prepare coating mixture of selexipag, mannitol and HPMC in hydro-alcoholic mixture
and apply it on the MCC spheres (pellets) using fluid bed coater.
2. Prepare coating mixture of ethyl cellulose and guar gum or ethyl cellulose and
hypromellose, and apply it on the step 1 prepared coated spheres (selexipag pellets) using
fluid bed coater.
3. Prepare coating mixture of selexipag, mannitol and HPMC in hydro-alcoholic mixture
and apply it on the step 2 prepared sustained release coated spheres (selexipag sustained
release pellets) using fluid bed coater to make bi-phasic controlled release pellets of
selexipag.
4. Step 3 pellets were filled in suitable size capsule and release was determined in 900
ml of phosphate buffer pH 6.8 vs. in 900 ml of 40% v/v ethanolic phosphate buffer pH
6.8 (360 mL of ethanol + 540 mL of phosphate buffer pH 6.8) using USP apparatus 2
(paddle) at 50 RPM and temperature of 37° ± 1° C.
5. Dissolution data of Example 4A:

| | Cumulative % w/w Release of Selexipag | |
|---|---|---|
| | | Media: 40% v/v |
| Time | Media: phosphate buffer | ethanolic phosphate |
| (hours) | pH 6.8 | buffer pH 6.8 |
| 1 | 23 | 25 |
| 2 | 52 | 61 |
| 4 | 72 | 80 |
| 8 | 88 | 93 |

| | |
|---|---|
| F2 Value | 59 |

6. Based on the obtained dissolution data, it was found that mixture of ethyl cellulose
and Hypromellose (HPMC) has high susceptibility to alcohol dose dumping, while the
mixture of ethyl cellulose and guar gum has very low susceptibility to alcohol dose
dumping, specifically when high viscosity guar gum is used as water soluble material.
The dissolution data revealed that the ratio of water-insoluble materials (such as ethyl
cellulose) to water-soluble materials (like guar gum) within the mixture of controlled
release excipients, along with their concentration in the pharmaceutical composition, is
critical for achieving the property of alcohol dose dumping.

What is claimed is:

1. A method for treating a pulmonary arterial hypertension comprising administering a controlled release pharmaceutical composition of selexipag or its active metabolite, wherein the said controlled release pharmaceutical composition releases the selexipag or its active metabolite in an amount of less than about 75% of the total amount of the selexipag or its active metabolite contained therein at 2 hours when measured in 900 ml of 40% v/v ethanolic phosphate buffer pH 6.8 using USP apparatus 2 at 50 RPM and temperature of 37°±1° C.,
wherein the said controlled release pharmaceutical composition comprises at least one water soluble material, optionally in combination with water insoluble material, in an amount of 2% w/w to about 93% w/w of the total weight of the pharmaceutical composition and is selected from the group of xanthan gum, acacia gum, diutan gum, tragacanth, gellan gum, guar gum, fenugreek gum, locust bean gum, pullulan, welan gum, polysaccharide, cellulose derivatives, and cellulose ether, wherein the said controlled release pharmaceutical composition comprises selexipag in an amount of 0.2 mg or more, wherein the said controlled release pharmaceutical composition release the selexipag or its active metabolite contained therein in an amount of less than about 60% at 2 hours but more than about 80% at 8 hours when measured in 900 ml of phosphate buffer pH 6.8 using USP apparatus 2 at 50 RPM and temperature of 37°±1° C.

2. A method for treating a pulmonary arterial hypertension of claim 1, wherein the controlled release pharmaceutical composition of selexipag or its active metabolite following oral administration with an alcoholic beverage provide a relative mean or median Cₘₐₓ of the selexipag or its active metabolite in a range of about 75 to 150% of the relative mean or median Cmax of the selexipag or its active metabolite, respectively, obtained following the oral administration of the same dose of the controlled release pharmaceutical composition of Selexipag or its active metabolite, respectively, with a non-alcoholic beverage.

3. A method for treating a pulmonary arterial hypertension of claim 1, wherein the controlled release pharmaceutical composition of selexipag or its active metabolite following oral administration with an alcoholic beverage provide a relative mean or median Tₘₐₓ of the selexipag or its active metabolite in a range of about 35 to 150% of the relative mean or median Tₘₐₓ of the selexipag or its active metabolite, respectively, obtained following the oral administration of the same dose of the controlled release pharmaceutical composition of Selexipag or its active metabolite, respectively, with a non-alcoholic beverage.

4. A method for treating a pulmonary arterial hypertension of claim 1, wherein the controlled release pharmaceutical composition of selexipag or its active metabolite following oral administration with an alcoholic beverage reduces incidence of at least one side effect associated with the selexipag or its active metabolite compared to the immediate-release dosage form comprising the same amount of the selexipag or active metabolite thereof.
